(19) United States
(12) Patent Application Publication
(10) Pub. No.: US 2010/0305673 A1
(43) Pub. Date: Dec. 2, 2010

(54) Ink Jet Printing of Implantable Electrodes

(75) Inventors: Claude Jolly, Innsbruck (AT); Stefan B. Nielsen, Innsbruck (AT)

Correspondence Address: Sunstein Kann Murphy & Timbers LLP, 125 SUMMER STREET, BOSTON, MA, 02110-1618, US

(73) Assignee: MED-EL ELEKTROMEDIZINISCHE GERAETE GMBH, Innsbruck (AT)

(21) Appl. No.: 12/787,866

(22) Filed: May 26, 2010

Related U.S. Application Data
(60) Provisional application No. 61/181,475, filed on May 27, 2009.

(51) Int. Cl. A61N 1/05; B05D 5/12; H05H 1/00; C23C 14/02

(52) U.S. Cl. 607/116; 427/77; 427/535; 427/534

(57) ABSTRACT

An implantable electrode device and a corresponding method of fabricating such a device are described. An electrode network of wires and contacts is developed by inkjet deposition of conductive metal material over portions of the electrode substrate for electrically connecting an implant processor device to targeted tissue in a patient. An electrode substrate beneath the electrode network provides structural support to the electrode network. A biocompatible encapsulation layer selectively covers a portion of the electrode network and provides electrical insulation for the covered portion of the electrode network while leaving exposed portions of the electrode network which allow electrical contact with adjacent tissue.

This application claims priority from U.S. Provisional Patent Application 61/181,475, filed May 27, 2009, incorporated herein by reference.

### FIELD OF THE INVENTION

The present invention relates to medical implants, and more specifically to a new type of implantable electrode for implant systems.

### BACKGROUND ART

Present day implantable electrodes such as cochlear implant electrodes require considerable amount of hand assembly during manufacturing. Individual thin platinum wires of about 25 μm in diameter with about 4 μm of polytetrafluoroethylene (PTFE) insulation must be cut to size and manipulated without compromising the insulation. The wires must be stripped of insulation at the ends and welded to thin platinum foils which have been cut to size, usually around 500 μm in diameter. Each individual wire must be placed one by one into a mold and assembled into a multi-channel structure before then being silicone injection molded. Demolding of the long electrodes then must take place without causing damage to the structure. There are some manufacturing rejects, for example, due to open or short circuits between wires, or poor welding of contacts. Silicone overflow onto the contact surfaces also cause some further rejects. The electrode making process is extremely labor intensive, a significant percentage of rejected electrodes is unavoidable, and maintaining adequate quality is problematic. Moreover, this highly manual work process is strongly operator dependent and it is difficult to specify in enough detail to ensure reproducible results. Hand made devices may therefore unintentionally be subject to significant variations in performance. Furthermore, the manual work is linked with extensive and time consuming training of personnel.

Semi-automated electrode fabrication processes can overcome some of the hurdles described above. Currently that typically involves photolithography and electroplating or vapor depositioning of metal (see e.g. WO2004064687, US2008027525, US2006017273; incorporated herein by reference) followed by thermal melt encapsulation or spin coating in an electrically insulating material to cover the conductive parts of the structure as needed to allow for adequate electrical stimulation. Although such semi-automated processes are precise and reproducible, they involve many individual process steps, some of which include extensive use of chemicals. Such chemicals may pollute materials that eventually are to be implanted, thus making purity control of chemicals and cleaning of electrodes very important factors. With existing processes it is possible to make structures in 2 dimensions—the height/thickness of the deposited metal is typically about the same at different depositioning locations—but it is not practical to make three dimensional structures because that would require physical masking of portions of the depositioning area and consequent interruption of the process.

Other semi-automated fabrication processes include removal of material from a sheet of metal to create predefined traces and pads (see e.g. U.S. Pat. No. 7,240,416, incorporated herein by reference, which suggests using embossing and electrical discharge machining or laser ablation). Embossing and selective material removal can facilitate making some three dimensional structures, but this may be limited by the thickness of the metal sheet used. Furthermore, it is generally not desirable to initially place a relatively large amount of metal (typically an expensive noble metal such as platinum or an alloy thereof) and then remove the majority of it for creating individual traces and pads.

### SUMMARY OF THE INVENTION

A method of producing an implantable electrode device starts by providing an electrode substrate for structural support. An electrode network of wires and contacts is developed over portions of the electrode substrate based on inkjet deposition of conductive metal material for electrically connecting an implant processor device to targeted tissue in a patient. A portion of the electrode network is selectively covered with a biocompatible encapsulation layer to provide electrical insulation for the covered portion of the electrode network, while also leaving exposed portions of the electrode network to allow electrical contact with adjacent tissue.

In further specific embodiments, the metal material may include platinum material such as a platinum-based ink and/or a platinum alloy material. For example, a metallic ink may contain metal nanoparticles or be based on a complex of platinum ions and surrounding ligands. Providing the electrode substrate may include initially treating the electrode substrate with at least one of a primer treatment and a plasma activation treatment to increase wettability of the metal material to the electrode substrate. Developing the electrode network also may include heat treating one or more portions of the electrode network for sintering. Developing the electrode network also may include developing selected portions of the electrode network to have a greater metal thickness than unselected portions of the electrode network. The greater metal thickness may include electroplated metal and/or inkjet deposited metal, and the selected portions may include selected exposed portions of the electrode network.

At least one of the substrate and the encapsulation layer may be formed of a silicone material. Providing the electrode substrate may include developing electrode channels by photo-resist processing for containing portions of the electrode network. The exposed portions of the electrode network are developed based on at least one of laser ablation, wet chemical removal, plasma etching, and mechanical treatment. Selectively covering a portion of the electrode network with the encapsulation layer may be based on at least one of spray coating, spin coating, inkjet printing, a thermal melting, and an injection molding process. The exposed portions may include one or more recessed portions wherein the exposed portion has a surface recessed below the surface of the adjacent encapsulant layer.

Developing the electrode network also may include inkjet printing the conductive metal material into recesses on the electrode substrate, which may be formed by embossment or injection molding in the electrode substrate. Developing the electrode network also may include inkjet printing the conductive metal material into recesses on a transfer plate, heat treating the conductive metal to form the electrode network, covering the electrode network with the electrode substrate, attaching the electrode substrate to the electrode network, and removing the electrode network and electrode substrate from the transfer plate.

Embodiments of the invention also include an implantable electrode device. An arrangement of conductive metal material is developed by inkjet deposition into an electrode network of wires and contacts for electrically connecting an implant processor device to targeted tissue in a patient. An electrode substrate beneath the electrode network provides structural support to the electrode network. A biocompatible encapsulation layer selectively covers a portion of the electrode network and providing electrical insulation for the covered portion of the electrode network, and leaves exposed portions of the electrode network to allow electrical contact with adjacent tissue.

In further specific such embodiments, the metal material may include platinum material such as a platinum-based ink. The metal material may be derived from a metallic ink containing metal nanoparticles or be based on a complex of platinum ions and surrounding ligands. The electrode substrate may include at least one of a primer treatment and a plasma activation treatment to increase wettability of the metal material to the electrode substrate. In other specific embodiments, one or more portions of the electrode network may be heat treated for sintering. The electrode network also may be developed so that selected portions of the electrode network have a greater metal thickness than unselected portions of the electrode network. The greater metal thickness may include electroplated metal and/or inkjet deposited metal, and the selected portions may include selected exposed portions of the electrode network.

The exposed portions of the electrode network may be developed based on at least one of laser ablation, wet chemical removal, plasma etching, and mechanical treatment. The encapsulation layer may be based on at least one of a spray coating, a spin coating, an inkjet printed coating, a thermal melted coating, and an injection molding process.

The exposed portions may include one or more recessed portions wherein the exposed portion has a surface recessed below the surface of the adjacent encapsulant layer. At least one of the substrate and the encapsulation layer may be formed of a silicone material. The electrode substrate may include electrode channels developed by photo-resist processing for containing portions of the electrode network. Or, the electrode network may be developed by inkjet printing the conductive metal material into recesses on the electrode substrate, which may be formed by embossment or injection molding in the electrode substrate. The electrode network also may be developed by inkjet printing the conductive metal material into recesses on a transfer plate, heat treating the conductive metal to form the electrode network, covering the electrode network with the electrode substrate, attaching the electrode substrate to the electrode network, and removing the electrode network and electrode substrate from the transfer plate.

### BRIEF DESCRIPTION OF THE DRAWINGS

FIG. 1 shows logical steps in manufacturing an implantable electrode device according to one embodiment.

FIG. 2 shows an example of an electrode network for an implantable electrode device which is inkjet printed on a substrate surface.

FIG. 3 shows an example of an electrode network of different heights using a single type of metal.

FIG. 4 shows an example of an electrode network of different heights using two different types of metal.

FIG. 5 shows an example of an electrode network printed on a substrate surface and partially covered by an encapsulation layer.

FIG. 6A-B shows an example of an electrode network printed into a recess in the substrate.

FIG. 7 shows an example of an electrode network of different heights printed into substrate recesses.

FIG. 8A-B shows examples of electrode networks printed into substrate recesses and partially covered by an encapsulation layer.

FIG. 9 shows an example using a photo-resist with substrate recesses to print an electrode network.

FIG. 10 A-D shows an embodiment developing an electrode network by inkjet printing conductive metal material into recesses on a temperature-resistant transfer plate.

### DETAILED DESCRIPTION OF SPECIFIC EMBODIMENTS

Various embodiments of the present invention are directed to a streamlined automated process to make implant electrode devices for neuro-stimulation which assembles platinum wires, electrode contacts and insulators in just a few steps, and with the ability to scale up as demand grows. Such an automated process can be implemented by adapting inkjet printing technology and metal-containing inks to inkjet print the electrode device onto a substrate (e.g. thin polymer film) to create a desired structure of conducting wires and stimulation contacts. Subsequent encapsulation of the printed electrode structures in electrically insulating polymer may then be done, for example, by thermal melting or spin coating. Such an inkjet printing process can be automated, flexible, comparably simple, and fast to ensure high reproducibility, thereby overcoming many of the challenges found in currently used and described alternative methods of manufacturing implantable electrode devices.

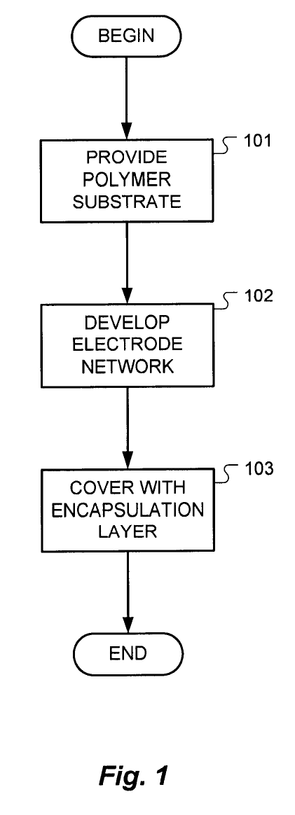

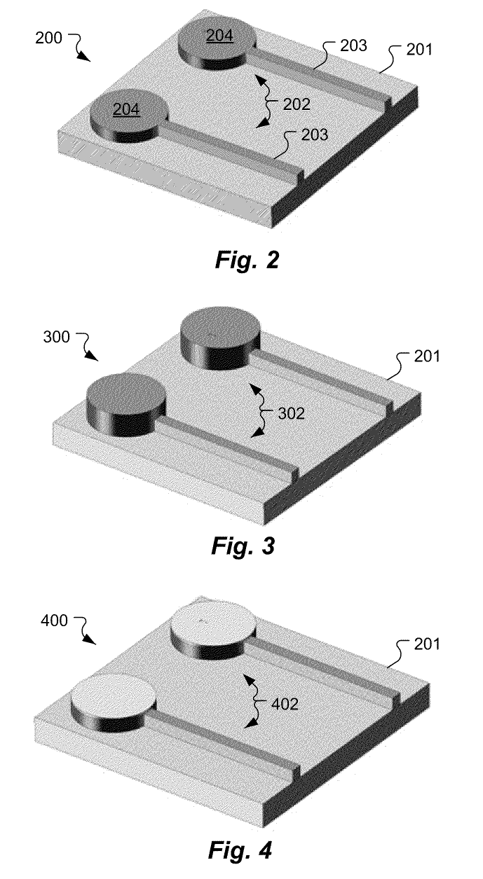

FIG. 1 shows the logical steps in manufacturing an implantable electrode device for electrically connecting an implant processor device to targeted tissue in a patient. And FIG. 2 shows an example of a corresponding electrode device 200 which is inkjet printed by the process. First, an electrode substrate 201 is provided, step 101, to establish structural support for the electrode device 200. Then, inkjet deposition of conductive metal material is used to develop an electrode network 202 of wires 203 and stimulation contacts 204 over portions of the electrode substrate 201, step 102. Due to the simplicity of the process, there is no need for aggressive chemicals that may pollute the electrode device 200, and biocompatibility issues are therefore largely related to the specific material used for the substrate 201 and to the specific ink formulation used to form the electrode network 202.

In one specific embodiment, the printed track of an individual wire 203 on the electrode substrate 201 typically has a width of about 100 μm and a height of a few hundred nm. These dimensions are in an interesting range for small multi-channel electrode devices 200 where multiple wires 203 and stimulation contacts 204 are used. The thin electrode network 202 enables the electrode device 200 to be highly flexible so that its mechanical properties are appropriate for in-vivo use such as, for example, cochlear implant electrode arrays that are inserted into the highly curved cochlea. On the other hand, very thin wires 203 can tend to have unduly high electrical resistance, which may be a problem due to the energy loss (resistance is a function of the cross-section area of the metal structure). To overcome this problem, several printing passes may be used to build up the material height of the wires 203 and thereby lower the electrical resistance.

In some specific embodiments, the conductive metal material of the electrode network 202 may include platinum material such as a platinum-based ink and/or a platinum alloy material. For example, a metallic ink may contain metal nanoparticles or be based on a complex of platinum ions and surrounding ligands. In some specific embodiments, the electrode substrate 201 may have been pre-treated with at least one of a primer treatment and a plasma activation treatment to increase wettability of the metal material to the electrode substrate 201. A metallic ink may not initially be conductive until it is sintered so as to either (depending on the ink formulation) fuse the platinum nanoparticles or cause reduction of the platinum complex in order to form solid metallic platinum. Thus, developing the electrode network 202 may include heat treating one or more portions of the electrode network 202 for sintering. FIG. 3 shows an example of an implantable electrode device 300 with an electrode network 302 having structural elements of different heights (e.g., the wires and stimulation contacts) using a single type of metal.

FIG. 4 shows another example of an implantable electrode device 400 with an electrode network 402 having structural elements of different heights using two different types of metal so that selected portions of the electrode network 402 have a greater metal thickness than unselected portions of the electrode network 402. The greater metal thickness may be developed based on electroplated metal and/or inkjet deposited metal. In some embodiments, and the selected portions with greater metal thickness may include selected exposed portions of the electrode network 402 (e.g., the electrode contacts).

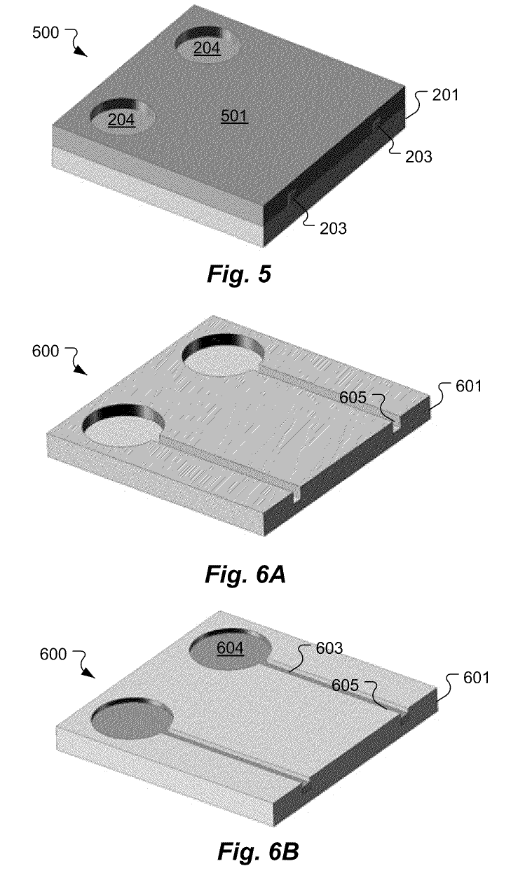

Once the electrode network 202 is developed, a portion of it is selectively covered with an encapsulation layer 501 in FIG. 5, step 103, made of an electrically non-conductive biocompatible material. The encapsulation layer 501 provides electrical insulation for the covered portion of the electrode network 202. The encapsulation layer 501 may be formed based on at least one of spray coating, spin coating, inkjet printing, a thermal melting, and an injection molding process. The selectively covering process needs to also leave some electrode contacts 204 exposed so as to allow electrical contact with adjacent tissue. Consequently, the exposed electrode contacts 204 may have a greater metal thickness than metal wires 203 beneath the encapsulation layer 501.

In the embodiment shown in FIG. 5, the exposed electrode contacts 204 of the implantable electrode device 500 are recessed below the surface of the adjacent encapsulant layer 501. For example, such recesses may be formed by injection molding of the encapsulation layer 501 or by embossing. In other embodiments, the exposed portions of the electrode network may be developed based on at least one of laser ablation, wet chemical removal, plasma etching, and mechanical treatment. At least one of the substrate 201 and the encapsulation layer 501 may be formed of a silicone material.

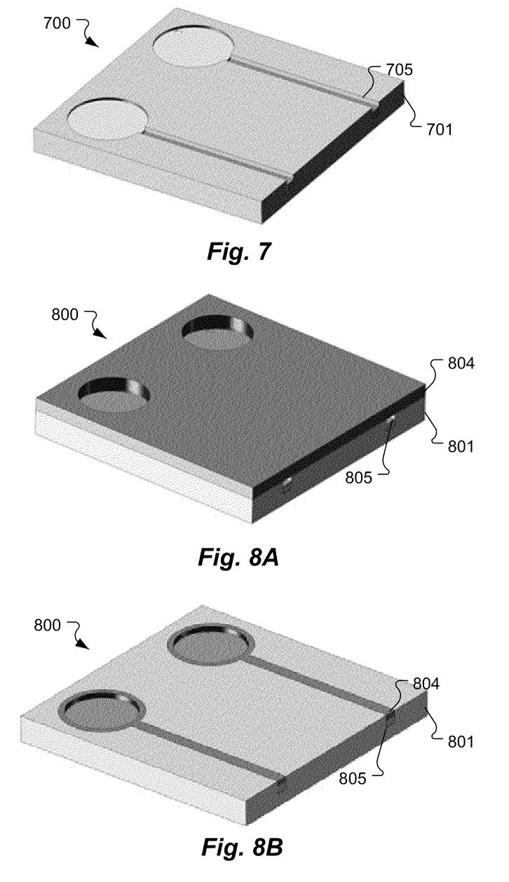

For some applications such as high-density electrodes or particularly small electrodes (e.g., cochlear implant electrodes), there may be problems related to spreading of the metallic ink over the surface of the substrate 201. If needed, the printability may be enhanced by matching the surface energy of the ink to the substrate 201 or by treating the surface of the substrate 201, for example, with a plasma treatment. Alternatively, FIGS. 6A and 6B show an embodiment of an implantable electrode device 600 wherein the electrode substrate 601 has recessed channels 605 into which the ink may be printed. Capillary forces will confine the ink to the recessed channels 605 to form the wires 603 and stimulation contacts 604. FIG. 7 shows an example of another embodiment an implantable electrode device 700 with an electrode network having metal structures of varying height which are printed into recessed channels 705 in the substrate 701 using two different types of metal. FIGS. 8A and 8B shown an implantable electrode device 800 where metal structures are printed into recessed channels 805 in the substrate 801 and then partially covered by an encapsulation layer 804 of electrically non-conductive biocompatible material.

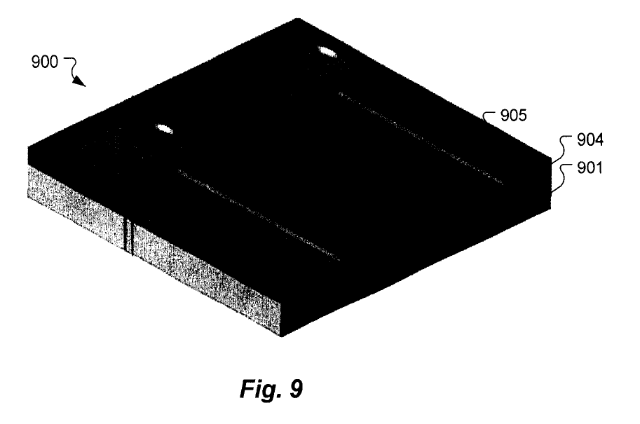

FIG. 9 shows an example of an implantable electrode device 900 based on using a photo-resist mask 904 having mask recesses 905 to print an electrode network. Standard photo-resist techniques may be used to provide the mask recesses 905 into which the metal structures of the electrode network are printed. The photo-resist mask 904 is then removed after drying and/or sintering of the metallic ink leaving an implantable electrode device 900 similar to the one shown in FIG. 2, but with better defined edges on the electrode network.

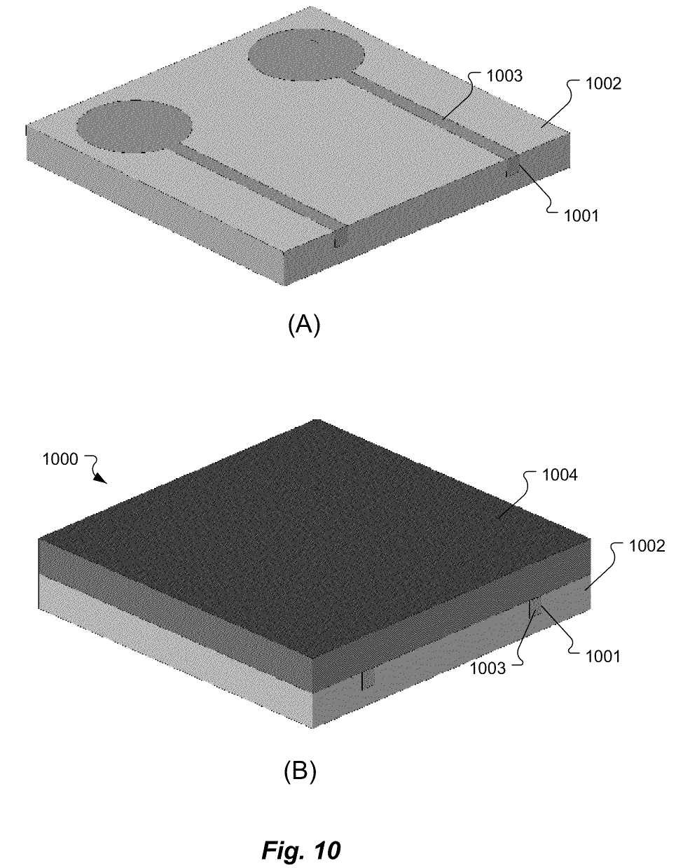

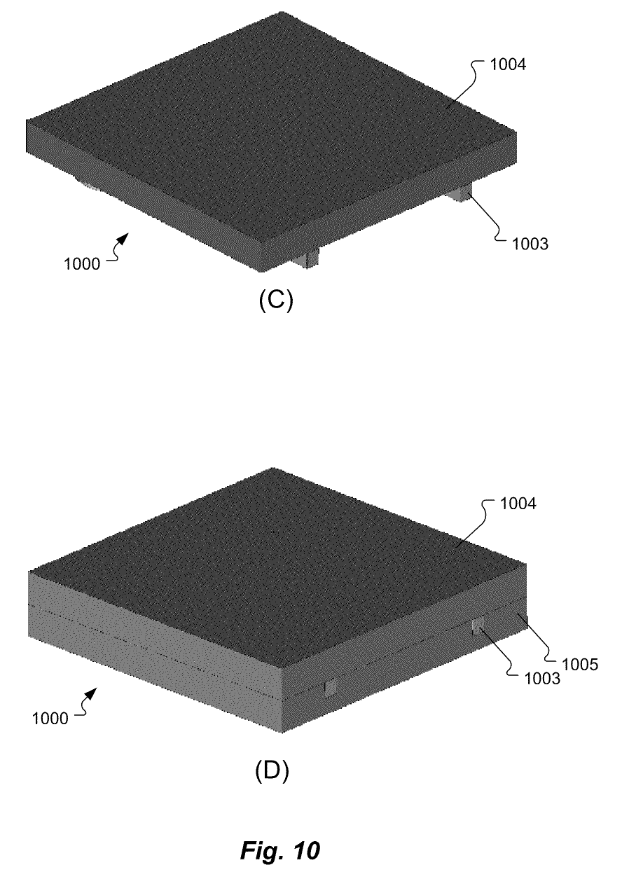

FIG. 10 A-D shows an embodiment wherein an implantable electrode device 1000 based on inkjet printing of conductive metal material into recesses 1001 on a temperature-resistant transfer plate 1002 (e.g. made of metal). The conductive metal 1003 is then heat treated with elevated temperatures to sinter the metal and evacuate any additive material from the deposited ink, thereby forming the electrode network 1003. Once the electrode network 1003 has been formed, it then may be covered with and attached to the electrode substrate 1004. The electrode network 1003 and electrode substrate 1004 can then be removed from the transfer plate 1002 and covered with an encapsulation layer 1005.

An inkjet printing process as described above to manufacture an implantable electrode device can be fast, simple, flexible, reproducible, and highly automated. Little complicated equipment is needed and there are relatively few process steps needed to make both two-dimensional and three-dimensional electrode network structures. And there is no need to waste expensive surplus material as in some other manufacturing methods since only the metal that is actually used for the electrode network is actually printed and used.

Structures using different metals can be produced in the same process by having ink cartridges with different inks. For example, in this way the electrode stimulation contacts can be coated or plated with a different metal than the bulk metal of the other portions of the electrode network. It is also possible to tailor the electrical and mechanical properties of the electrode device in a single process by printing structures that include elements which serve a mechanical purpose without necessarily being electrically active. For example, structures of non-conductive materials can be printed in the same manufacturing process by using printing cartridges with an appropriate material (e.g. a structural polymer). This also allows printing of structures for electrical insulation.

In addition, changing the design parameters of the manufactured structures is relatively simple with only simple changes in a CAD file where the geometrical parameters for the electrode design are defined and possibly some basic process optimization. In a case of printing into recessed geometries, the process for creating the recesses may also need to be changed.

For more background information on inkjet printing in embossed polymer structures refer to C. E. Hendriks, P. J. Smith, J. Perelaer, A. M. J. van den Berg, U. S. Schubert, Invisible Silver Tracks Produced by Combining Hot-Embossing and Inkjet Printing, Adv. Funct. Mater., 18, 1031-1038, (2008), incorporated herein by reference.

Although various exemplary embodiments of the invention have been disclosed, it should be apparent to those skilled in the art that various changes and modifications can be made which will achieve some of the advantages of the invention without departing from the true scope of the invention.

What is claimed is:

1. A method of producing an implantable electrode device, the method comprising:
providing a electrode substrate for structural support;
developing an electrode network of wires and contacts by inkjet deposition of conductive metal material over portions of the electrode substrate for electrically connecting an implant processor device to targeted tissue in a patient; and
selectively covering a portion of the electrode network with a biocompatible encapsulation layer for providing electrical insulation for the covered portion of the electrode network, wherein the selectively covering leaves a plurality of exposed portions of the electrode network to allow electrical contact with adjacent tissue.

2. A method according to claim 1, wherein the metal material includes platinum material.

3. A method according to claim 2, wherein the platinum material is derived from a platinum-based ink.

4. A method according to claim 3, wherein the platinum-based ink is based on a complex of platinum ions and surrounding ligands.

5. A method according to claim 1, wherein the metal material is derived from a metallic ink containing metal nanoparticles.

6. A method according to claim 1, wherein providing the electrode substrate includes:
initially treating the electrode substrate with at least one of a primer treatment and a plasma activation treatment to increase wettability of the metal material to the electrode substrate.

7. A method according to claim 1, wherein developing the electrode network includes heat treating one or more portions of the electrode network for sintering.

8. A method according to claim 1, wherein developing the electrode network includes developing selected portions of the electrode network to have a greater metal thickness than unselected portions of the electrode network.

9. A method according to claim 8, wherein the greater metal thickness includes electroplated metal.

10. A method according to claim 8, wherein the greater metal thickness includes inkjet deposited metal.

11. A method according to claim 8, wherein the selected portions include selected exposed portions of the electrode network.

12. A method according to claim 1, wherein the exposed portions of the electrode network are developed based on at least one of laser ablation, wet chemical removal, plasma etching, and mechanical treatment.

13. A method according to claim 1, wherein selectively covering portion of the electrode network with the encapsulation layer is based on at least one of spray coating, spin coating, inkjet printing, and a thermal melting.

14. A method according to claim 1, wherein selectively covering portion of the electrode network with the encapsulation layer is based on an injection molding process.

15. A method according to claim 1, wherein the exposed portions include one or more recessed portions wherein the exposed portion has a surface recessed below the surface of the adjacent encapsulant layer.

16. A method according to claim 1, wherein at least one of the substrate and the encapsulation layer is formed of a silicone material.

17. A method according to claim 1, wherein providing the electrode substrate includes developing a plurality of electrode channels by photo-resist processing for containing portions of the electrode network.

18. A method according to claim 1, wherein developing the electrode network includes:
inkjet printing the conductive metal material into a plurality of recesses on the electrode substrate.

19. A method according to claim 18, wherein the one or more recessed portions are formed by embossment or injection molding in the electrode substrate.

20. A method according to claim 1, wherein developing the electrode network includes:
inkjet printing the conductive metal material into a plurality of recesses on a transfer plate;
heat treating the conductive metal to form the electrode network;
covering the electrode network with the electrode substrate;
attaching the electrode substrate to the electrode network; and
removing the electrode network and electrode substrate from the transfer plate.

21. An implantable electrode device comprising:
an arrangement of conductive metal material developed from inkjet deposition into an electrode network of wires and contacts for electrically connecting an implant processor device to targeted tissue in a patient;
a electrode substrate beneath the electrode network and providing structural support to the electrode network; and
a biocompatible encapsulation layer selectively covering a portion of the electrode network and providing electrical insulation for the covered portion of the electrode network, and leaving a plurality of exposed portions of the electrode network which allow electrical contact with adjacent tissue.

22. A device according to claim 21, wherein the metal material includes platinum material.

23. A device according to claim 22, wherein the platinum material is derived from a platinum-based ink.

24. A device according to claim 23, wherein the platinum-based ink is based on a complex of platinum ions and surrounding ligands.

25. A device according to claim 21, wherein the metal material is derived from a metallic ink containing metal nanoparticles.

26. A device according to claim 21, wherein the electrode substrate includes at least one of a primer treatment and a plasma activation treatment to increase wettability of the metal material to the electrode substrate.

27. A device according to claim 21, wherein one or more portions of the electrode network are heat treated for sintering.

28. A device according to claim 21, wherein selected portions of the electrode network have a greater metal thickness than unselected portions of the electrode network.

29. A device according to claim 28, wherein the greater metal thickness includes electroplated metal.

30. A device according to claim 28, wherein the greater metal thickness includes inkjet deposited metal.

31. A device according to claim 28, wherein the selected portions include selected exposed portions of the electrode network.

32. A device according to claim 21, wherein the exposed portions of the electrode network are developed based on at least one of laser ablation, wet chemical removal, plasma etching, and mechanical treatment.

33. A device according to claim 21, wherein the encapsulation layer is based on at least one of a spray coating, a spin coating, an inkjet printed coating, and a thermal melted coating.

34. A device according to claim 21, wherein the encapsulation layer is derived from an injection molding process.

35. A device according to claim 21, wherein the exposed portions include one or more recessed portions wherein the exposed portion has a surface recessed below the surface of the adjacent encapsulant layer.

36. A device according to claim 21, wherein at least one of the substrate and the encapsulation layer is formed of a silicone material.

37. A device according to claim 21, wherein the electrode substrate includes a plurality of electrode channels developed by photo-resist processing for containing portions of the electrode network.

38. A device according to claim 21, wherein the electrode network is developed by inkjet printing the conductive metal material into a plurality of recesses on the electrode substrate.

39. A device according to claim 38, wherein the one or more recessed portions are formed by embossment or injection molding in the electrode substrate.

40. A device according to claim 21, wherein the electrode network is developed from inkjet printing the conductive metal material into a plurality of recesses on a transfer plate.
